(19) United States
(12) Patent Application Publication
(10) Pub. No.: US 2003/0051989 A1
(43) Pub. Date: Mar. 20, 2003

(54) Method to liberate hydrocarbon fractions from hydrocarbon mixtures

(75) Inventor: Douglas P. Austin, Salt Lake City, UT (US)

Correspondence Address: M. Wayne Western, THORPE, NORTH & WESTERN, L.L.P., P.O. Box 1219, Sandy, UT, 84091-1219, US

(73) Assignee: Petronetics, LLC.

(21) Appl. No.: 10/174,304

(22) Filed: Jun. 17, 2002

Related U.S. Application Data
(60) Provisional application No. 60/299,107, filed on Jun. 18, 2001.

(51) Int. Cl. C07C 1/00; C07C 2/00; C07C 4/00; C07C 5/00; C07C 6/00; C07F 1/00; C07F 3/00; C07F 5/00; C07B 31/00

(52) U.S. Cl. 204/157.15; 204/157.6; 204/157.62

(57) ABSTRACT

A method of liberating various existing hydrocarbon fractions from hydrocarbon mixtures without the need of additives, catalysts or heating using ultrasonic cavitation. Ultrasonic energy is provided at a rate sufficient to induce cavitation in the hydrocarbon mixture without causing cracking. The high temperatures and high pressures resulting from cavitation reduce intermolecular forces between hydrocarbons thereby liberating existing lighter hydrocarbons in the diesel range or lighter for recovery via more traditional separation technologies. The resulting upgraded petroleum product exhibits lower distillation curves and decreased pollution causing components. Further, a wide variety of feedstocks can be treated according to the method of this invention.

### CROSS-REFERENCE TO RELATED APPLICATIONS

This application claims the benefit of U.S. Application No. 60/299,107, filed Jun. 18, 2001.

### BACKGROUND OF THE INVENTION

1. Field of the Invention

This invention relates to petroleum mixtures, and, more specifically, to hydrocarbon mixtures and a method for separating various hydrocarbon fractions from the same.

2. Related Art

The commercial and household products that are derived from crude oil are almost too numerous to mention. Petroleum products are used in the manufacture of goods utilized in residential and commercial construction, automobiles, fibers for clothing, holiday decorations, food processing and packaging, medical devices, and the synthesis of pharmaceuticals. The route from crude oil to sweaters, CD's, car bumpers, roofing shingles, etc., is a long one involving refining and reforming. The products which can be derived from an average barrel of crude oil, which contains 42 gallons, include gasoline to power our vehicles; kerosene used as a jet fuel and used around the world for cooking and space heating; liquefied petroleum gas (LPG) used as fuel and as an intermediate material in the manufacture of petrochemicals; diesel fuels and domestic heating oils; residual fuels or combinations of residual and distillate fuels for heating and processing; coke used as briquets; asphalt used for roads and roofing materials; solvents such as benzene, toluene, and xylene; petrochemical feedstocks used in the production of plastics, synthetic fibers, synthetic rubbers, and other products; and lubricating oil base stocks such as motor oils, industrial greases, lubricants, and cutting oils.

High-grade crudes which directly produce large amounts of gasoline have the most commercial value. Those which need considerable conversion to produce significant amounts of gasoline or contain larger than usual amounts of metals such as vanadium (which poisons or shortens the life of the catalysts used in reforming) have the lowest dollar value. The average crude, after refining, typically yields an approximate product mixture shown below in Table 1.

TABLE 1
Average product mixture of refined crude oil.

| Refinery Product | Hydrocarbon Range | Percent |
|---|---|---|
| Gasoline | C₅-C₁₀ | 27 |
| Kerosene | C₁₁-C₁₈ | 15 |
| Diesel | C₁₄-C₁₉ | 11 |
| Heavy Gas Oil | C₁₂-C₂₅ | 10 |
| Lubricating Oil | C₂₀-C₄₀ | 20 |
| Residuum | >C₄₀ | 17 |

While there are direct markets for the lighter fuels (gasoline, kerosene, and diesel), in order to be profitable the other components of the crude oil, especially the gas oil and residuum, need to be converted into marketable products. This is the role of catalytic cracking. Further, about 70% of crude oil used in the United States undergoes some type of conversion process. An overview of common petroleum refining processes is shown below in Table 2.

TABLE 2
Overview of Petroleum Refining Processes

| | | | | | |
|---|---|---|---|---|---|
| Process name | Action | Method | Purpose | Feedstock(s) | Product(s) |

Fractionation Processes

| | | | | | |
|---|---|---|---|---|---|
| Atmospheric | Separation | Thermal | Separate fractions | Desalted crude oil | Gas, gas oil, |
| distillation | | | | | distillate, residual |
| Vacuum | Separation | Thermal | Separate w/o | Atmospheric | Gas oil, lube |
| distillation | | | cracking | tower residual | stock, residual |

Conversion Processes - Decomposition

| | | | | | |
|---|---|---|---|---|---|
| Catalytic | Alteration | Catalytic | Upgrade gasoline | Gas oil, coke | Gasoline, |
| cracking | | | | distillate | petrochemical |
| | | | | | feedstock |
| Coking | Polymerize | Thermal | Convert vacuum | Residual, heavy | Naptha, gas oil, |
| | | | residuals | oil, tar | coke |
| Hydrocracking | Hydrogenate | Catalytic | Covert to oil, | Gas oil, cracked | Lighter, higher- |
| | | | lighter HCs | residual | quality products |
| Hydrogen steam | Decompose | Thermal/cat. | Produce hydrogen | Desulfurized gas, | Hydrogen, CO, |
| reforming | | | | O₂, steam | CO₂ |
| Steam cracking | Decompose | Thermal | Crack large | Atm. tower heavy | Cracked naptha, |
| | | | molecules | fuel/distillate | coke, residual |
| Visbreaking | Decompose | Thermal | Reduce viscosity | Atmospheric | Distillate, tar |
| | | | | tower residual | |

Conversion Processes - Unification

| | | | | | |
|---|---|---|---|---|---|
| Alkylation | Combining | Catalytic | Unite olefins & | Tower isobutane/ | Iso-octane |
| | | | amp; isoparaffins | crckr olefin | (alkylate) |
| Grease | Combining | Thermal | Combine soaps & | Lube oil; fatty | Lubricating |
| compounding | | | amp; oils | acid; alky metal | grease |
| Polymerization | Polymerize | Catalytic | Unite 2 or more | Cracker olefins | High-octane |
| | | | olefins | | naptha, |
| | | | | | petrochemical |
| | | | | | stocks |

Conversion Processes - Alteration or Re-arrangement

| | | | | | |
|---|---|---|---|---|---|
| Catalytic | Alteration/ | Catalytic | Upgrade low- | Coker/hydro- | High-octane |
| reforming | dehydration | | octane | cracker | reformate |

Despite the various processes for converting petroleum, the industry still suffers from an inability to efficiently convert heavy hydrocarbon fuels into lighter, more valuable hydrocarbons. Current methods of catalytic cracking of heavy hydrocarbon fuels are expensive, inefficient, and require large amounts of capital investment. Current methods also produce less than desirable results because cracking is random and unpredictable. Heavy hydrocarbons containing high concentrations of trace metals, such as vanadium, cause fouling of most common catalysts so as to preclude catalytic cracking. Further, many vacuum gas oils contain lighter fractions which, when catalytically cracked, produce excess amounts of gases and undesirable by-products.

The petroleum industry has explored many avenues for reducing these problems. Among these avenues is the use of sonic and ultrasonic energy in a variety of applications. Most frequently ultrasonic energy is used in conjunction with various carrier agents, such as surfactants and other emulsifying agents, to cause scission of carbon-carbon bonds in various petroleum mixtures. Most methods involve the use of an emulsifying agent, catalyst, or a combination of the two among a variety of processing methods.

Crude oil is comprised of hydrocarbon fractions of varying chain lengths, as seen in Table 1. The longer chain lengths have progressively higher boiling points, and therefore the varying chain lengths can be separated out by distillation. In a typical oil refinery, crude oil is progressively heated and the constituent components are largely vaporized according to their boiling points corresponding to the pressure existing in the column at that point. The various components may then be drawn from the column at points of differing temperatures and pressures. The heavier fractions recovered, such as heavy lubricating oils and residuums, generally have significantly less commercial value than the lighter fractions.

Other methods involve the use of ultrasound on intentionally created oil-in-aqueous phase emulsions in the presence of a catalyst. These types of methods crack heavier hydrocarbons to produce lighter more valuable products with the added expense of creating and controlling the emulsion composition, often complex additives, and a catalyst.

Therefore, there remains a need in the art for an efficient and cost-effective method of upgrading hydrocarbon mixtures that does not require additives such as water or other catalysts, does not require the formation of an emulsion, and does not require heating the hydrocarbon mixtures prior to or during the upgrading process. Additionally, there is a need for a method to improve the ability to separate various hydrocarbon fractions existing in hydrocarbon mixtures which are not recovered using traditional distillation and separation processes.

### SUMMARY OF THE INVENTION

This invention makes possible the liberation of various hydrocarbon fractions previously left unrecovered in various hydrocarbon mixtures. Treatment using the method of the present invention can improve the separability of various hydrocarbon fractions from crude oils and other hydrocarbon mixtures. By treating hydrocarbon mixtures with energy sufficient to cause cavitation this invention also has utility for the production of more valuable hydrocarbon products from what were previously considered very low value hydrocarbon mixtures. As such, a completely new source of feedstock for the production of valuable petroleum products can be made available for use by petroleum refiners to expand the range of feedstock currently available.

The method of the present invention involves liberating various existing hydrocarbon fractions from a hydrocarbon mixture. The hydrocarbon mixture is treated with cavitational energy sufficient to weaken and disrupt or eliminate molecular attractions between the molecules of the hydrocarbon mixture without causing cracking. The various hydrocarbon fractions may then be separated using any number of separation technologies, most often distillation.

In another aspect of the present invention the cavitational energy may be provided using ultrasonic, electromagnetic, propeller, impeller, venturi methods, or combinations thereof.

An advantage of the method of the present invention is that a wide variety of hydrocarbon mixtures can be used as feedstock. Non-limiting examples include crude oil, atmospheric tower refining bottoms, used motor oil, vacuum gas oils, refining residuums, fuel oil, vacuum tower bottoms, residual fuel oils and mixtures of these feedstocks.

In another more detailed aspect of the present invention the hydrocarbon mixture further includes components containing nitrogen, sulfur, chlorine or oxygen.

In another more detailed aspect of the present invention the hydrocarbon mixture is treated at a temperature less than about 20° F. over the pour point of the mixture.

In yet another more detailed aspect of the present invention the hydrocarbon mixture is treated in the absence of a substantial aqueous phase or additives.

In one more detailed embodiment of the invention a cup-shaped flow tube is used to direct flow of the hydrocarbon mixture toward the ultrasonic energy source and accelerate flow to the turbulent flow regime.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 is a block diagram showing the method steps of the process of the present invention for applying cavitational energy to treat hydrocarbon mixtures;

FIG. 2 is a schematic diagram showing a system for treating hydrocarbon mixtures according to one embodiment of the present invention; and

FIG. 3 is a schematic diagram showing one possible flow configuration past an ultrasonic energy source of the system shown in FIG. 2.

### DETAILED DESCRIPTION OF THE INVENTION

### A. DEFINITIONS

In conjunction with the disclosure herein, the following terms will be used as defined, unless otherwise specified or made clear in the context used.

As used herein, “hydrocarbon fuel”, “hydrocarbon mixture” and “hydrocarbon product” are used interchangeably and refer to any petroleum or hydrocarbon mixture such as crude oil, used motor oil, vacuum gas oils, refining residuums, fuel oil, vacuum tower bottoms, atmospheric tower refining bottoms, residual fuel oils and mixtures thereof. Frequently, the hydrocarbon product has previously undergone more traditional separation and/or distillation processes or is a residual product of other processes. Further, many hydrocarbon mixtures of interest also contain complex mixtures of heterocyclic and heteroatom hydrocarbon compounds, aromatics, cyclic hydrocarbons, trace elements and hydrocarbons having non-carbon constituent groups which include but are not limited to sulfur, oxygen, nitrogen, chlorine and various combinations of these. Examples of such compounds include but are not limited to quinolines, pyrrols, cresols, alcohols and phenols.

As used herein, “hydrocarbon fraction” is intended to refer generally to a portion of a hydrocarbon mixture which, if isolated, exhibits abounded range of boiling points at a given pressure distinct from the remainder of the hydrocarbon mixture or other existing hydrocarbon fractions. This definition includes both hydrocarbon fractions which may not actually distill prior to treatment according to the present invention and those fractions which distill without treatment.

As used herein, “cavitation” refers to the result of stresses induced in a liquid by the passing of a sound wave through the liquid. A sound wave consists of compression and decompression/rarefaction cycles. These waves may be produced by a variety of methods such as when an alternating current voltage is applied to a crystal, the crystal expands and contracts in phase with the electric field according to the piezoelectric effect, or expansion and contraction of a magnetorestrictive alloy. These cavitation bubbles (similar to those seen arising from the action of a boat propeller on water) are at the heart of ultrasonic cavitation or sonochemistry systems. This series of sound wave cycles cause the bubbles to grow during a decompression phase, and contract or implode during a compression phase when they reach a critical size. Thus the temperatures and pressures upon implosion after reaching the critical size is related to the frequency and intensity of the sound waves. Each one of these imploding bubbles can therefore be seen as a microreactor, with temperatures reaching over an estimated 5000° C., and pressures of over several hundred atmospheres. Cavitation is therefore the production of cavities or bubbles in a fluid using ultrasound followed by an implosion of the cavity.

As used herein, “cavitational energy” refers to energy which is sufficient to cause cavitation to occur in a liquid. The cavitational energy may be provided using various methods known to those skilled in the art.

As used herein, “disruption” of intermolecular and intramolecular forces refers to the reduction, weakening, prevention, inhibition, eliminating or any lessening of the attractive forces between atoms of molecules which effects are more than transient in duration. This disruption may be the result of physical and/or chemical changes which reduce attractive forces between molecules of a fluid or different sites of the same molecule.

As used herein, the “pour point” of a fluid is the lowest temperature at which a fluid is observed to flow, when cooled under conditions prescribed by test method ASTM D 97. The pour point is 3° C. (5° F.) above the temperature at which the fluid in a test vessel shows no movement when the container is held horizontally for five seconds.

### B. METHOD OF SEPARATING FRACTIONS FROM A HYDROCARBON MIXTURE

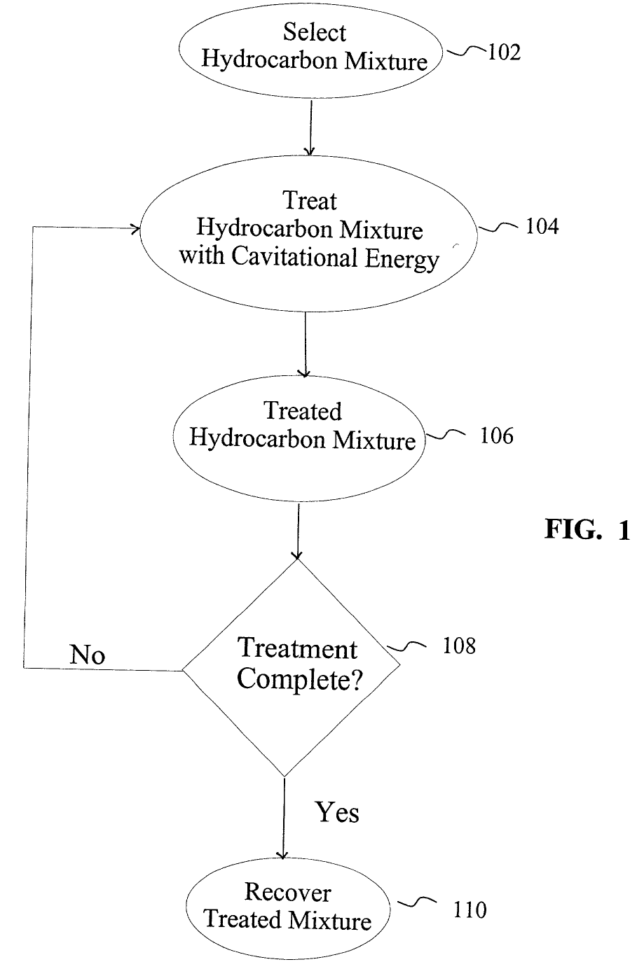

Referring now to FIG. 1, hydrocarbon mixtures 102 are selected for treatment to improve their utility and value. As shown in FIG. 1, the selected hydrocarbon mixtures 102 are then processed via a system for treating with cavitational energy 104 which results in an treated hydrocarbon mixture 106 containing a higher content of distillable and more valuable recoverable hydrocarbons. The lighter hydrocarbons may then be recycled for further treatment at step 108 or recovered and separated at step 110 from the heavier hydrocarbons using traditional techniques such as distillation. Although ultrasonic methods offer many benefits in providing cavitational energy such as space, cost and efficiency, other methods of causing cavitation could be used in the method of the present invention. These other methods include but are not limited to propellers, impellers, venturi, electromagnetic waves, or any other method sufficient to cause cavitation of the hydrocarbon mixture.

The hydrocarbon mixtures 102 may include a broad range of hydrocarbon containing mixtures. Non-limiting examples of hydrocarbon mixtures which may benefit from application of the present invention are crude oil, atmospheric tower refining bottoms, used motor oil, vacuum gas oils, refining residuums, fuel oils, vacuum tower bottoms, residual fuel oils, #6 fuel oils and mixtures of these hydrocarbons. These hydrocarbon mixtures often contain lighter hydrocarbons that do not distill during traditional separations processes. Further, as mentioned earlier hydrocarbon mixtures and petroleum products in particular contain a complex mixture of straight chain hydrocarbons, branched and cyclic hydrocarbons, aromatics, heterocyclic compounds and often include various non-carbon-containing constituent groups. It is the presence of these heterocyclic and heteroatom compounds that often cause problems in traditional refining processes such as fouling and discoloring and require hydrotreating or use of additional processes to remove or reduce these effects. Treatment of commercial gasoline, #1 diesel fuel, #2 diesel fuel or mixtures of these hydrocarbons improves combustion and affects a reduction in emission of toxic unburned hydrocarbons and reduced formation of NOx during combustion.

One important aspect of the present invention is the absence of the requirement to add additional agents prior to treatment. However, it should be noted that the presence of additives or an aqueous phase does not preclude use of the present invention. Those skilled in the art will recognize that some feedstocks may require pretreatment to remove troublesome components, however the process has proven very versatile and no pretreatment is normally required. “Additives”, as used herein, is not intended to include components normally found in the subject feedstock or are added during prior processing or use. Treatment of crude oil in accordance with the present invention prior to the distillation process will increase the yields of lighter hydrocarbon fractions and reduce the need for further processing such as cracking or other upgrading. Treatment of #6 fuel oil according to the method of the present invention produces both diesel boiling range fractions and the residual is a high quality asphalt product.

The hydrocarbon mixture does not require heating for practice of the present invention and may even be practiced at ambient temperatures or below. Although not required for practice of the present invention, the mixture can be heated to allow flow to occur. Frequently the mixture will be pumped through a continuous system which requires a degree of flowability in the feedstock. Temperatures below about 300° F. typically provide the desired flowability and temperatures less than about 20° F. above the pour point of the fluid should suffice for most applications of the present invention.

Another advantage of the present invention is that, because ultrasonic cavitation equipment is significantly less expensive than thermal or catalytic cracking equipment, processing of small volume streams of hydrocarbon mixtures is economically feasible. Another advantage of the invention is that the method produces no substantial environmental emissions or off gases. Further, the method is a totally self-contained process which may be easily moved to different locations and occupies minimal space. Conversion cost is also very low requiring minimal electrical energy. Another advantage of the present invention is that the method can be performed without requiring the formation of emulsions either before or during the process of exposing the hydrocarbon mixture to ultrasonic energy.

Referring again to FIG. 1, the method steps in accordance with the present invention begins by selecting 102 an appropriate hydrocarbon mixture for treatment. Typically, the process of the present invention is applied to petroleum or hydrocarbon mixtures having at least two hydrocarbon fractions. Once the hydrocarbon mixture is selected, processing continues to the cavitational energy treatment step 104. At this step, the hydrocarbon mixture is treated by applying cavitational energy wherein the hydrocarbon mixture is directly exposed to cavitational energy. The preferred system for applying cavitational energy is described in greater detail below and one embodiment is described hereinafter. When using ultrasonic cavitational energy sources, it is desirable that the sound waves cycle at a rate sufficient to induce cavitation and implosion of the cavitation cavities in the hydrocarbon mixture and disrupt molecular attractions between the molecules of the hydrocarbon mixture and minimize heteroatom interference without causing substantial cracking of the hydrocarbon mixture. Any frequency which is functional to obtain the desired disruption without also cracking molecules of the hydrocarbon mixture is acceptable for practice of the present invention. Sound waves having a frequency of about 5 kHz to about 500 kHz are useful. Frequencies from about 10 kHz to about 50 kHz are readily commercially available, while a frequency of about 18 kHz to about 22 kHz has proven particularly effective in practice of the present invention.

The exposure time varies and is a function of the flow rate of the hydrocarbon mixture past the ultrasonic energy source, e.g., an ultrasonic horn 306. Exposure is limited to avoid causing substantial cracking of the feedstock, therefore less than 375 W/cm² is required although exposure up to 500 W/cm² could be used if cracking is avoided. Further, exposure in the range of less than about 100 W/cm² has typically offered good results. Other ultrasonic energy sources may be used in accordance with the present invention such as magnetorestrictive alloys, such as terfenol, or any other ultrasonic generators known to those skilled in the art. As mentioned earlier, other sources may produce the energy needed to produce cavitation within the hydrocarbon mixture. These cavitational energy sources include not only ultrasonic horns and probes, but also propellers, impellers, venturi, electromagnetic waves and combinations of these sources.

In one embodiment of the present invention an ultrasonic horn is used as the cavitational energy source and the hydrocarbon mixture is directed past the ultrasonic horn in a continuous process. The hydrocarbon mixture is provided at a flow rate which depends on the quality and viscosity of the feedstock but may vary from about 2 to about 20 gallons per minute while a flow rate of about 5 to about 15 gallons per minute for a 1.5″ ultrasonic horn yields good results. Clearly, the addition of flow cells configured to direct flow past the cavitational energy source will allow for increased flow rates without negatively affecting the process efficiency. Further discussion of the flow past the ultrasonic energy source is provided in more detail below in relation to the “cup-shaped” flow tube.

Although not generally necessary, the hydrocarbon mixture may be recycled through the cavitational treatment step as shown in step 108. The treated hydrocarbon mixture can be tested at this point and recycled until the desired characteristics are achieved. Alternatively, instead of continuously feeding a hydrocarbon mixture past an ultrasonic horn, a fixed amount of hydrocarbon mixture may be placed in a container along with ultrasonic energy inducing probes in a batch process. A batch treatment according to this method would be particularly suited for mixtures containing highly viscous hydrocarbons, residuums or heavy waxes but is less efficient than continuous flow processing.

The chemical effects of ultrasound are to enhance reaction rates because of the formation of highly reactive radical species formed during cavitation and the disruption of intermolecular and intramolecular attractive forces. The method of the present invention affects a reduction in van der Waals, polar attractive forces, hydrogen bonding and other attractive forces as a result of both physical and/or chemical changes.

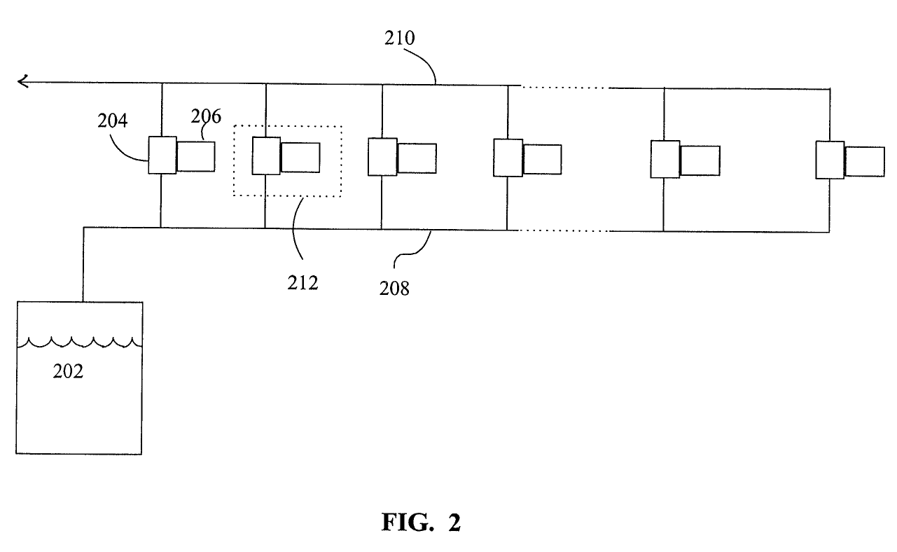

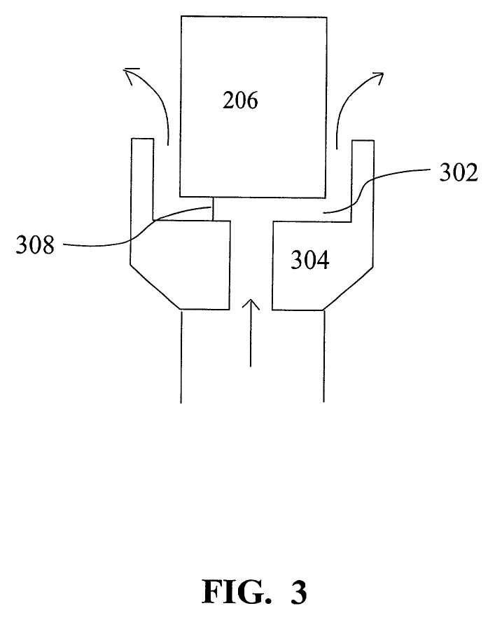

While various methods of generating sound waves are known in the art, such as a sonic transducer with a magnetorestrictive alloy, the currently preferred method uses ultrasonic horns containing piezo-electric crystals as the ultrasonic energy source 206, shown in FIG. 2. The hydrocarbon mixture is delivered to the ultrasonic energy source using any number of flow cell 204 configurations which define a containment space and direct the flow of fluid for exposure to the ultrasonic energy. A particularly effective flow cell for delivering the hydrocarbon mixture to the ultrasonic energy source is shown in FIG. 3. A “U” or cup-shaped flow tube 304 is placed to direct the flow of feedstock approaching the ultrasonic horns 206. The cup-shaped flow tube, due to its reduced diameter and “U” shape, enhances the effectiveness of the system. It is thought that this improved performance is the result of increasing the velocity of the feedstock resulting in turbulent, rather than laminar flow, as the feedstock approaches the ultrasonic horns. Several variables seem to affect the efficiency of the process and include the gap 308 between the flow tube and the ultrasonic energy source, and the cupped walls on the flow tube. Tests performed using a flow tube without the cupped walls showed a reduced effect on the distillation of the treated hydrocarbon mixture. Further, the gap should be adjusted to that which is functional to obtain the desired results. A narrow gap produces undesirable emulsions while a slightly larger gap will affect the desired results and requires minimal experimentation to determine. For example, a configuration having a gap of ⅜″, an inlet diameter of ⅜″, and an ultrasonic horn diameter of ¾″ is one operable configuration. The resulting turbulent flow and high pressures cause more of the feedstock to come into close contact with the ultrasonic horns resulting in increased cavitation of the feedstock. The flow tube also directs the feedstock across the full diameter of the ultrasonic horn and increases the exposure of the fluid to cavitational energy. The cup-shaped flow tube 304, as used in one embodiment of the present invention, advantageously and unexpectedly increases the cavitation of the hydrocarbon mixtures used as feedstock thereby increasing the effectiveness of the process. Further, under laminar flow conditions without a cup-shaped flow tube increasing the flow rate of a sample of used motor oil from 3 to 5 gpm resulted in poorer distillation results. However, the addition of the cup-shaped flow tube resulted in similar distillation results at 5 gpm as the 3 gpm tests without the flow tube. Thus, the cupped walls of the flow tube provide more favorable conditions for separating the various hydrocarbon fractions than without. Further, flow rates at about 10 gpm have also shown good results using this flow tube for a variety of feedstocks.

A cup-shape flow tube which is effective in providing the discussed results is a commercially available product available as a 1.5″ high pressure process cell assembly and is available in a range of sizes. Using the 1.5″ flow tube and the above configuration produces an exposure of between about 40 W/cm² and 100 W/cm² when using a 1000W energy supply. Other flow tubes or delivery systems directing flow toward the cavitational energy source wherein the flow is provided in the turbulent flow regime will also improve the effectiveness of the cavitational energy treatment. Such tubes and systems include also introducing obstructions or any change in diameter or flow-direction which would cause increased turbulent flow and mixing of the delivered feedstock.

Clearly, the optimal flow rate past the ultrasonic horns will depend on a variety of factors such as feedstock viscosity, temperature, composition and flow tube characteristics delivering feedstock past the ultrasonic horns. Feedstocks containing highly viscous components will require lower flow rates or repeated exposure to cavitational energy.

Frequently heavy hydrocarbon products of various processes such as atmospheric tower bottoms, residuums, asphalts and #6 fuel oil contain significant amounts of lighter hydrocarbons, i.e. the diesel fuel range and lighter, which failed to separate out during previous traditional processing. There are a variety of factors that lead to lighter hydrocarbons remaining unrecovered in hydrocarbon products, such as incomplete distillation and poor processing. It is also thought that a significant amount of lighter hydrocarbons remain trapped in the mixture because of intermolecular and intramolecular interactions and strong attractive forces among the molecules of the hydrocarbon mixture. Application of the method of the present invention to this type of mixture tends to decrease these attractive forces between hydrocarbons to allow the lighter hydrocarbons to be recovered and used as a higher value hydrocarbon product. It is important to note that treatment according to the method of the present invention results in a lasting effect on the hydrocarbon mixture. The treated mixture may be stored or shipped without recovering the liberated hydrocarbon fractions and the later performed separation exhibits essentially the same improvements in distillation yields as separations performed immediately after treatment with cavitational energy. Storage of treated mixtures for over six months has resulted in minimal or no loss of the improvement in distillation of hydrocarbon fractions.

Referring again to FIG. 1, after the hydrocarbon mixture is treated by the cavitational energy in step 104, processing continues to step 108 wherein the system determines whether the treatment is complete. For efficient processing, the hydrocarbon mixture should reach a predefined fractionation value. If at step 108 the predefined threshold has not been reached, processing returns to step 104 for treatment with additional cavitational energy. If step 108 determines that the predefined threshold, such as fractionation value, has been reached, processing continues to step 110. Most often a single pass through the system is sufficient if the optimal conditions are chosen as discussed previously.

### C. EXAMPLES

### General Experimental Testing Procedures

Unless otherwise indicated the following test equipment was used in each example: sonochemical horn (20 kHz), sonochemical power supply (1000 W), process cell and ASTM D-86 Atmospheric Distillation Test Apparatus. All percents shown are by volume unless otherwise indicated. Temperatures for each example before treatment were between about 50° F. and about 300° F. and were typically within 10° to 20° F. above the pour point of the hydrocarbon mixture. Mere flowability of the hydrocarbon mixture was required to perform the method of the present invention.

### Example 1

A 100 ml sample of each of two separate crude oil samples were tested for initial ASTM D-86 Atmospheric Distillation values as shown in Table 3. In separate tests, a gallon of each of the crude oils was then placed in a continuous flow test bed where cavitation was then introduced by the ultrasonic horn into the sample. The samples were then re-tested for ASTM D-86 Atmospheric Distillation results which are shown in Table 3.

TABLE 3
ASTM D-86 Atmospheric Distillation Results (° F.)

| | Crude Oil 1 | | Crude Oil 2 | |
|---|---|---|---|---|
| | Before | After | Before | After |
| % Recovered | cavitation | cavitation | cavitation | cavitation |
| Initial bp | 130 | 115 | 114 | 108 |
| 5% | 172 | 128 | 152 | 141 |
| 10% | 218 | 176 | 215 | 189 |
| 20% | 307 | 252 | 260 | 289 |
| 30% | 403 | 340 | 392 | 406 |
| 40% | 492 | 432 | 497 | 502 |
| 50% | 570 | 508 | 592 | 582 |
| 60% | 634 | 576 | 638 | 600 |
| 70% | 670 @ 67% | 623 | 649 | 617 |
| 80% | | 655 | 670 @ 74% | 624 |
| 90% | | 662 @ 86% | | 670 @ 88% |
| 95% | | | | |

These results show a significant increase in the yield of fractions boiling under 670° F. Crude oil 1 resulted in approximately 19% increase, while crude oil 2 resulted in a 14% increase in yield. This represents a substantial benefit, since the fractions boiling in this range can be used as diesel fuel or similar products having a greater value than the residual.

### Example 2

A 100 ml sample of each of Canadian Bow River, Canadian CMS and Southwest Wyoming Light Sweet crude oils were tested for initial ASTM D-86 Atmospheric Distillation values as shown in Table 4. In separate tests, a gallon of each of the crude oils was then placed in a continuous flow test bed where cavitation was then introduced by the ultrasonic horn into the sample. The samples were then re-tested for ASTM D-86 Atmospheric Distillation results which are shown in Table 4.

TABLE 4
ASTM D-86 Atmospheric Distillation Results (° F.)

| | | |
|---|---|---|
| Bow River | CMS | Wyoming |

| | Before | After | Before | After | Before | After |
|---|---|---|---|---|---|---|
| % Recovered | cavitation | cavitation | cavitation | cavitation | cavitation | cavitation |
| Initial bp | 98 | 115 | 111 | 109 | 92 | 101 |
| 5% | 150 | 142 | 154 | 150 | 143 | 126 |
| 10% | 222 | 190 | 195 | 176 | 165 | 159 |
| 20% | 396 | 320 | 261 | 256 | 196 | 203 |
| 30% | 546 | 492 | 351 | 349 | 221 | 225 |
| 40% | 602 | 520 | 460 | 445 | 247 | 248 |
| 50% | 640 | 569 | 540 | 532 | 281 | 285 |
| 60% | 662 | 575 | 610 | 601 | 330 | 340 |
| 70% | | 630 | | 645 | 411 | 425 |
| 80% | | 638 | | 662 | 528 | 507 |
| 90% | | | | | 701 | 650 |
| 95% | | | | | | |

Application of ultrasonic energy to the Wyoming sample resulted in minimal change in distillation values. This sample is a lighter crude oil as compared to the Canada crudes. This suggests that the heavier crude oils exerted greater intermolecular forces to prevent the lighter fractions from distilling than in the Wyoming crude oil. These results also show a significant increase in the yield of fractions boiling under about 670° F. for the Canadian crudes, each improving yield by as much as 20%.

### Example 3

A sample of used motor oil was tested for initial ASTM D-86 Atmospheric Distillation values as shown in Table 5. The sample was continuously fed through an ultrasonic processing system, similar to the arrangement shown in FIG. 3. The table shows results at various flow rates and with and without the U-shaped flow tube. The sample was then re-tested for ASTM D-86 Atmospheric Distillation results which are shown in Table 5.

TABLE 5
ASTM D-86 Atmospheric Distillation Results (° F.)

| | | | |
|---|---|---|---|
| | | No U-shape | U-shape |
| % Recovered | No processing | Flow tube | Flow tube |

| — | — | 3 gpm | 5 gpm | 5 gpm | 10 gpm |
|---|---|---|---|---|---|
| Initial boiling | 330 | 318 | 324 | 311 | 330 |
| point | | | | | |
| 5% | 485 | 370 | 477 | 379 | 399 |
| 10% | 660 | 455 | 595 | 462 | 448 |
| 20% | 710 | 542 | 657 | 591 | 557 |
| 30% | 732 | 588 | 702 | 627 | 605 |
| 40% | 740 | 612 | 716 | 650 | 638 |
| 50% | 762 | 622 | 727 | 663 | 656 |
| 60% | 812 | 628 | 735 | 681 | 663 |
| 70% | 840 | 638 | | 696 | 692 |
| 80% | 868 | 655 | | 718 | 725 |
| 90% | 914 | | | | |
| 95% | 950 | | | | |

These results show a very significant increase in the yield of fractions boiling under 670° F. The untreated used motor oil under 700° F. was just under 20%, while after treatment the four tests resulted in between about 15% and 60% increase in yield. Notice that the non-cup-shaped flow tube gave good results, while the cup-shaped flow tube allowed for a higher flow rate and improved yields.

### Example 4

A 100 ml sample of vacuum gas oil was tested for initial ASTM D-86 Atmospheric Distillation values as shown in Table 6. A gallon of the vacuum gas oil was then placed in a continuous flow test bed where cavitation was then introduced by the ultrasonic horn into the sample. The sample was then re-tested for ASTM D-86 Atmospheric Distillation results which are shown in Table 6.

TABLE 6
ASTM D-86 Atmospheric Distillation Results (° F.)

| % Recovered | Before cavitation | After cavitation |
|---|---|---|
| Initial boiling point | 345 | 255 |
| 5% | 407 | 319 |
| 10% | 483 | 345 |
| 20% | 560 | 520 |
| 30% | 610 | 546 |
| 40% | 640 | 576 |
| 50% | 656 | 602 |
| 60% | 675 @ 59% | 623 |
| 70% | | 640 |
| 80% | | 648 @ 77% |

These results show a significant increase in the yield of fractions boiling under 670° F. The treatment resulted in about a 31% increase in yield at about 650° F. This represents a substantial benefit, since the fractions boiling in this range can be used as diesel fuel or similar products having a greater value than the original vacuum gas oil.

### Example 5

A 100 ml sample of atmospheric tower bottoms was tested for initial ASTM D-86 Atmospheric Distillation values as shown in Table 7. A gallon of the atmospheric tower bottoms was then placed in a continuous flow test bed where cavitation was then introduced by the ultrasonic horn into the sample. The sample was then re-tested for ASTM D-86 Atmospheric Distillation results which are shown in Table 7.

TABLE 7
ASTM D-86 Atmospheric Distillation Results (° F.)

| % Recovered | Before cavitation | After cavitation |
|---|---|---|
| Initial boiling point | 330 | 221 |
| 5% | 518 | 329 |
| 10% | 568 | 410 |
| 20% | 607 | 556 |
| 30% | 622 | 588 |
| 40% | 635 | 616 |
| 50% | 659 | 628 |
| 60% | 663 @ 58% | 638 |
| 70% | | 652 |
| 80% | | 656 @ 73% |

These results show a significant increase in the yield of fractions boiling under about 660° F. The treatment resulted in about a 15% increase in yields at this temperature.

### Example 6

A 100 ml sample of vacuum tower bottoms was tested for initial ASTM D-86 Atmospheric Distillation values as shown in Table 8. A gallon of the vacuum tower bottoms was then placed in a continuous flow test bed where cavitation was then introduced by the ultrasonic horn into the sample. The sample was then re-tested for ASTM D-86 Atmospheric Distillation results which are shown in Table 8.

TABLE 8
ASTM D-86 Atmospheric Distillation Results (° F.)

| % Recovered | Before cavitation | After cavitation |
|---|---|---|
| Initial boiling point | 635 | 540 |
| 5% | 670 @ 3% | 595 |
| 10% | | 610 |
| 20% | | 640 |
| 30% | | 665 |
| 40% | | 670 @ 32% |
| 50% | | |

Again, these results show a very significant increase in the yield of fractions boiling under 670° F. The treatment resulted in about a 29% increase in yield at about 670° F. Remember that the starting material was vacuum tower bottoms and such improvement represents a dramatic increase in diesel boiling range products having a greater value than the original.

### Example 7

A 100 ml sample each of gasoline and #2 diesel fuel were tested for initial ASTM D-86 Atmospheric Distillation values as shown in Table 9. In separate tests, a gallon of gasoline and #2 diesel fuel were then placed in a continuous flow test bed where cavitation was then introduced by the ultrasonic horn into the sample. The samples were then re-tested for ASTM D-86 Atmospheric Distillation results which are shown in Table 9.

TABLE 9
ASTM D-86 Atmospheric Distillation Results (° F.)

| | Gasoline | | Diesel | |
|---|---|---|---|---|
| | Before | After | Before | After |
| % Recovered | cavitation | cavitation | cavitation | cavitation |
| Initial bp | 91 | 89 | 302 | 179 |
| 5% | 109 | 99 | 338 | 307 |
| 10% | 117 | 106 | 361 | 345 |
| 20% | 133 | 121 | 387 | 380 |
| 30% | 144 | 136 | 417 | 410 |
| 40% | 171 | 176 | 461 | 453 |
| 50% | 191 | 192 | 475 | 463 |
| 60% | 206 | 206 | 508 | 498 |
| 70% | 229 | 225 | 529 | 508 |
| 80% | 273 | 262 | 541 | 527 |

Although less dramatic than the other examples, commercially available gasoline and diesel can also benefit from application of the present invention. Although this results in small increases in the yield of fractions boiling at various temperatures, initial indications show improvement in the combustion characteristics of both fuels. This could result in lower emissions and less NOx produced during combustion.

The test data from these multiple examples supports the following conclusions. First, ultrasonic cavitation treatment according to the present invention results in an upgraded product having a greater portion of distillable lighter and more valuable hydrocarbon fractions than the original feed stock. Second, ultrasonic cavitation allows extant lighter hydrocarbons to distill closer to their normal boiling points. Third, insubstantial cracking occurs in performance of the method of the present invention. The process does not cause coke formation, liberate off gases, nor cause a change in odor normally present in thermal or catalytic cracking. Further, there is no apparent volumetric increase or change in API gravity which would indicate the occurrence of cracking. Test data suggest that an average of 40 to 60% by volume of the hydrocarbon mixtures are liberated into more valuable fuel fractions. Practice of the present invention therefore provides for liberating or release of smaller molecules which are not distillable by traditional refining technologies and offers the industry a new tool to maximize the yield of valuable hydrocarbon fractions.

### D. SYSTEM FOR APPLYING ULTRASONIC ENERGY TO HYDROCARBON MIXTURES

A system of the present invention for applying ultrasonic energy to hydrocarbon mixtures and generating a treated hydrocarbon product having more distillable lighter hydrocarbons is shown in FIG. 2. In the embodiment shown in FIG. 2, the system for applying ultrasonic energy shown is a continuous feed system. The hydrocarbon mixture 202 is continuously fed through an incoming feed line 208 which is operatively connected to one or more ultrasonic sub-systems 212. The number of sub-systems will depend on the desired capacity and maybe arranged in series or parallel based on basic process design principles for either processing or reliability factors. Although a plurality of ultrasonic sub-systems are shown in FIG. 2 only a single ultrasonic sub-system is labeled for convenience. Once treatment is complete, the treated hydrocarbon mixture, or the fuel having a higher distillable hydrocarbon content, is removed from the ultrasonic sub-system(s) 212 of the system through a processed product return line 210.

A sample ultrasonic sub-system 212 is shown in FIG. 3. In this particular embodiment, the hydrocarbon mixture enters the flow cell 204 which defines a containment space directing the flow of the hydrocarbon mixture. The ultrasonic sub-system applies ultrasonic energy to the hydrocarbon mixture by using an ultrasonic energy source 206. One embodiment of the flow cell 204 is the “U” or cup-shaped flow tube 304 depicted in FIG. 3 and is particularly effective in delivering the hydrocarbon mixture to the ultrasonic horn although other flow cells and configurations would suffice for practice of the present invention. The gap 308 between the ultrasonic energy source and the flow tube 304 is an experimentally determined distance and may depend on a variety of factors. For the configuration shown where the flow tube inlet is ⅜″ diameter and the ultrasonic energy source is 1.5″ diameter, a gap of ⅜″ provides adequate results while a gap of ¼″ creates undesirable emulsions. Therefore, the appropriate configurations require some minor experimentation to determine and are well within the capacity of those skilled in the art.

The manner in which the flow is directed past the ultrasonic horn directly affects the efficiency of the treatment process and care should be taken to provide for maximum exposure of the fluid across the surface of the ultrasonic horn. The treated hydrocarbon mixture exits the ultrasonic sub-system 212 via the processed product return line 210. The treated hydrocarbon mixture may then be stored or processed further via distillation or other refining processes.

The system, including the ultrasonic sub-systems, are described in these terms for convenience purposes only. In addition, the components of the system described herein are commercially available wherein it is well known by a person of ordinary skill in the relevant art to design, implement, and operate such a system in order to perform the method of separating various hydrocarbon fractions from a hydrocarbon mixture according to the present invention.

### CONCLUSION

While various embodiments of the present invention have been described above, it should be understood that they have been presented by way of example only, and not limitation. It will be understood by those skilled in the art that various changes in form and details may be made therein without departing from the spirit and scope of the invention as defined in the specification and the appended claims. Thus, the breadth and scope of the present invention should not be limited by any of the above-described exemplary embodiments, but should be defined in accordance with the specification and any equivalents.

What is claimed is:

1. A method of liberating various existing hydrocarbon fractions from a hydrocarbon mixture, comprising the steps of:
(a) providing a hydrocarbon mixture, wherein the hydrocarbon mixture comprises at least two hydrocarbon fractions; and
(b) treating the hydrocarbon mixture with cavitational energy, wherein the cavitational energy induces cavitation in the hydrocarbon mixture insufficient to cause substantial cracking of hydrocarbons within the hydrocarbon mixture yet sufficient to disrupt molecular attractions between molecules of the hydrocarbon mixture to produce a treated hydrocarbon mixture.

2. The method of claim 1, wherein the cavitational energy is provided using a cavitational energy source selected from the group consisting of ultrasonic, electromagnetic, propeller, impeller, venturi and combinations thereof.

3. The method of claim 2, wherein the cavitational energy source is an ultrasonic source.

4. The method of claim 3, wherein the ultrasonic source comprises one or more ultrasonic horns.

5. The method of claim 3, wherein the cavitational energy has a frequency of about 5 kHz to about 500 kHz.

6. The method of claim 5, wherein the cavitational energy has a frequency of about 18 kHz to about 22 kHz.

7. The method of claim 3, wherein the cavitational energy is less than about 375 W/cm².

8. The method of claim 1, wherein the hydrocarbon mixture is selected from the group consisting of crude oil, atmospheric tower refining bottoms, used motor oil, vacuum gas oils, refining residuums, fuel oil, vacuum tower bottoms, residual fuel oils and mixtures thereof.

9. The method of claim 1, wherein the hydrocarbon mixture is selected from the group consisting of commercial gasoline, #1 diesel fuel, #2 diesel fuel and mixtures thereof.

10. The method of claim 1, wherein the hydrocarbon mixture further comprises organic components containing heteroatoms selected from the group consisting of nitrogen, sulfur, chlorine, oxygen and mixtures thereof.

11. The method of claim 1, wherein the hydrocarbon mixture is treated at a temperature less than about 300° F.

12. The method of claim 1, wherein the hydrocarbon mixture having a predetermined pour point is treated at a temperature less than about 20° F. over the pour point of the hydrocarbon mixture.

13. The method of claim 1, wherein the hydrocarbon mixture is treated in the absence of substantial amounts of an aqueous phase or additives.

14. The method of claim 1, further comprising the step of separating the at least two hydrocarbon fractions from the treated hydrocarbon mixture into separate fractions.

15. The method of claim 14, wherein the step of separating is accomplished via distillation.

16. The method of claim 1, further comprising subjecting the treated hydrocarbon mixture to step (b) multiple times until the treated hydrocarbon mixture exhibits a predetermined fractionation value.

17. A treated hydrocarbon mixture produced by the method of claim 1, wherein the treated hydrocarbon mixture has a higher distillable hydrocarbon content than the original hydrocarbon mixture.

18. A method of liberating various existing hydrocarbon fractions from a hydrocarbon mixture, comprising the steps of:
(a) providing a hydrocarbon mixture in the absence of substantial amounts of an aqueous phase or additives, wherein the hydrocarbon mixture comprises at least two hydrocarbon fractions;
(b) treating the hydrocarbon mixture with ultrasonic energy, wherein the ultrasonic energy induces cavitation in the hydrocarbon mixture insufficient to cause substantial cracking of hydrocarbons within the hydrocarbon mixture yet sufficient to disrupt molecular attractions between molecules of the hydrocarbon mixture to produce a treated hydrocarbon mixture; and
(c) separating the at least two hydrocarbon fractions from the treated hydrocarbon mixture into separate fractions.

19. A method of liberating various existing hydrocarbon fractions from a hydrocarbon mixture, comprising the steps of:
(a) providing a hydrocarbon mixture in the absence of substantial amounts of an aqueous phase or additives, wherein the hydrocarbon mixture comprises at least two hydrocarbon fractions;
(b) treating the hydrocarbon mixture with ultrasonic energy, wherein the ultrasonic energy induces cavitation in the hydrocarbon mixture insufficient to cause substantial cracking of hydrocarbons within the hydrocarbon mixture yet sufficient to disrupt molecular attractions between molecules of the hydrocarbon mixture to produce a treated hydrocarbon mixture and wherein the hydrocarbon mixture further has a predetermined pour point and is treated at a temperature from the pour point of the hydrocarbon mixture to about 20° F. over the pour point; and
(c) separating the at least two hydrocarbon fractions from the treated hydrocarbon mixture into separate fractions.

20. A method of liberating various existing hydrocarbon fractions from a hydrocarbon mixture, comprising the steps of:
(a) providing a hydrocarbon mixture, wherein the hydrocarbon mixture comprises at least two hydrocarbon fractions;
(b) providing a means for delivering the hydrocarbon mixture to a source of ultrasonic energy using a cup-shaped flow tube;
(c) treating the hydrocarbon mixture with ultrasonic energy, wherein the ultrasonic energy induces cavitation in the hydrocarbon mixture insufficient to cause substantial cracking of hydrocarbons within the hydrocarbon mixture yet sufficient to disrupt molecular attractions between molecules of the hydrocarbon mixture to produce a treated hydrocarbon mixture.

21. A continuous self-contained ultrasonic treatment system for liberating various existing hydrocarbon fractions from hydrocarbon mixtures comprising:
(a) a containment space for containing the hydrocarbon mixture;
(b) an inlet line operatively connected to the containment space;
(c) at least one ultrasonic energy source for emitting ultrasonic energy positioned such that the ultrasonic energy passes through the containment space;
(d) an outlet line operatively connected to the containment space to allow for withdrawal of the hydrocarbon mixture; and
(e) a cup-shaped flow tube operatively connected to the containment space and oriented to direct flow of the hydrocarbon mixture toward the ultrasonic energy source.

22. A method of liberating various existing hydrocarbon fractions from a hydrocarbon mixture, comprising the steps of:
(a) providing a hydrocarbon mixture wherein the hydrocarbon mixture is substantially free of an aqueous phase and additives and is at a temperature less than 20° F. over the pour point of the hydrocarbon mixture and wherein the hydrocarbon mixture comprises at least two hydrocarbon fractions;
(c) directing the hydrocarbon mixture toward an ultrasonic energy source using a cup-shaped flow tube;
(b) treating the hydrocarbon mixture with ultrasonic energy emitted from the ultrasonic energy source, wherein the ultrasonic energy has sufficient energy to disrupt molecular attractions between molecules of the hydrocarbon mixture but insufficient energy to cause substantial cracking of hydrocarbons within the hydrocarbon mixture to produce a treated hydrocarbon mixture; and
(c) recovering the at least two hydrocarbon fractions from the treated hydrocarbon mixture.
